(19) United States
(12) Patent Application Publication
(10) Pub. No.: US 2025/0228472 A1
(43) Pub. Date: Jul. 17, 2025

(54) SYSTEM AND METHOD TO AUTOMATICALLY ACTIVATE ANALYTE MONITORS

(71) Applicant: LAXMI THERAPEUTIC DEVICES, INC., Goleta, CA (US)

(72) Inventors: Thomas Metzmaker, Goleta, CA (US); Shane A. Williams, Santa Barbara, CA (US); Timothy J. Uhl, Goleta, CA (US)

(21) Appl. No.: 19/025,478

(22) Filed: Jan. 16, 2025

Related U.S. Application Data
(60) Provisional application No. 63/621,905, filed on Jan. 17, 2024.

(51) Int. Cl. A61B 5/145; A61B 5/00

(52) U.S. Cl. CPC A61B 5/14503; A61B 5/14546; A61B 5/6832; A61B 2560/0214; A61B 2560/0443; A61B 2560/0462

(57) ABSTRACT

A method of operating an analyte monitor including a sensor, a transmitter, and a battery, includes inserting the sensor under a patient's skin, and connecting the analyte monitor with an external device. Operation of the analyte monitor is automatically activated after the sensor is inserted under the patient's skin and prior to the analyte monitor being connected with the external device.

### CROSS-REFERENCE TO RELATED APPLICATION(S)

The present application claims priority to and the benefit of U.S. Provisional Patent Application No. 63/621,905, filed Jan. 17, 2024, the contents of which are hereby incorporated by reference in their entirety.

### BACKGROUND

### Field

The present disclosure relates to monitors attachable to a subject's body, for measuring analytes, such as glucose, in the body of the subject. The present disclosure further relates to methods of activating such monitors upon application to the subject.

### Description of Related Art

Monitoring different analytes in the human body can used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to simplify activation procedures for such monitors to, for example, make activation easier for end users and/or to prevent user error when initially applying and activating such monitors. This may be particularly useful for continuous glucose monitors, which may be attached to the patient's body for a prolonged period of time, where errors in initial application and activation may impact monitor readings for large portions of the activation time of such continuous glucose monitors. Furthermore, more robust activation protocols may also allow for useful information on the patient to be collected earlier in the operation period of the monitor. Such benefits may generally lead to more effective operation of such monitors.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensor of the glucose monitor under the patient's skin. Thereafter, there is generally an activation step that is taken to electronically activate the sensor of the glucose monitor and to facilitate communication between the sensor and the other electrical components of the glucose monitor. These steps may further facilitate powering up of the electrical components of the monitor, and/or other initiation steps to facilitate proper functionality of the monitor.

An aspect of one or more embodiments of the present disclosure is directed towards an analyte monitor including a sensor configured to be inserted under a patient's skin, a housing configured to be adhered to a surface of the patient's skin, a transmitter for transmitting information associated with the sensor, and a battery for providing power to the analyte monitor. The analyte monitor is not electrically activated prior to the sensor being inserted under the patient's skin and is automatically electrically activated after the sensor is inserted under the patient's skin.

The transmitter may be separable from the housing. The battery may be housed in the housing. The patient may be configured to assemble the transmitter to the housing, and the analyte monitor may be automatically electrically activated upon assembly of the transmitter to the housing. The battery may be housed in the housing, and the automatic electrical activation may include electrically connecting the battery to the transmitter to provide power to the transmitter.

The housing may hold the battery and at least part of the sensor, while the transmitter may be configured to be spaced apart from the housing.

The automatic electrical activation may be configured to be effected absent any further actions by the patient.

An aspect of one or more other embodiments of the present disclosure may be directed towards a method of operating an analyte monitor including a sensor, a transmitter, and a battery, where the method includes inserting the sensor under a patient's skin, and connecting the analyte monitor with an external device. Operation of the analyte monitor is automatically activated after the sensor is inserted under the patient's skin and prior to the analyte monitor being connected with the external device.

The operation of the analyte monitor may be automatically activated absent any explicit activation step by the patient.

The connection may be a wireless connection.

The analyte monitor may be separated into at least two parts, and wherein the method further comprises assembly of the at least two parts together. A first part of the at least two parts may include a housing that holds the battery and at least part of the sensor, and a second part of the at least two parts may include the transmitter. The assembly of the at least two parts may close an electrical connection between at least the battery and the transmitter. The assembly of the at least two parts may further electrically connect the transmitter with the sensor. The second part including the transmitter may be configured to be detached from the first part and to be reused with a first part of a different analyte monitor.

The operation of the analyte monitor may include collecting data from the sensor prior to the analyte monitor being connected with the external device. The analyte monitor may further include storage for storing the collected data from the sensor prior to the analyte monitor being connected with the external device. The collected data may be delivered to the external device after the analyte monitor is connected with the external device.

### BRIEF DESCRIPTION OF THE DRAWINGS

Further features and advantages of the invention will be come apparent from the description of embodiments by means of the accompanying drawings. In the drawings:

FIGS. 1A and 1B schematically show a human body with a monitor including an analyte sensor according to embodiments of the invention, where the monitor is attached at different positions on the body.

FIG. 2 shows a perspective view from above an exemplary monitor including an analyte sensor according to embodiments of the invention.

FIG. 3 shows a perspective from below the monitor of FIG. 2.

FIG. 4A shows a perspective view from above a cradle of an exemplary monitor, for example, the monitor of FIGS. 2 and 3.

FIG. 4B shows a perspective view from above a transmitter of an exemplary monitor, for example, the monitor of FIGS. 2 and 3.

FIG. 5 shows a side view of another exemplary monitor including an analyte sensor according to embodiments of the invention.

FIG. 6 schematically shows a transmitter and a sensor carrier or other cradle of an exemplary monitor prior to the two components being assembled together.

FIG. 7 schematically shows the transmitter and the sensor carrier or other cradle of the exemplary monitor of FIG. 6 after assembly.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

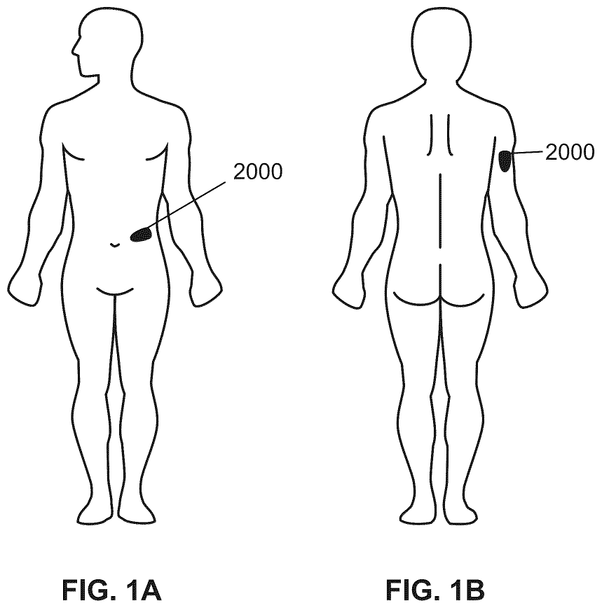

Monitors that include analyte sensors, such as glucose monitors, and particularly continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

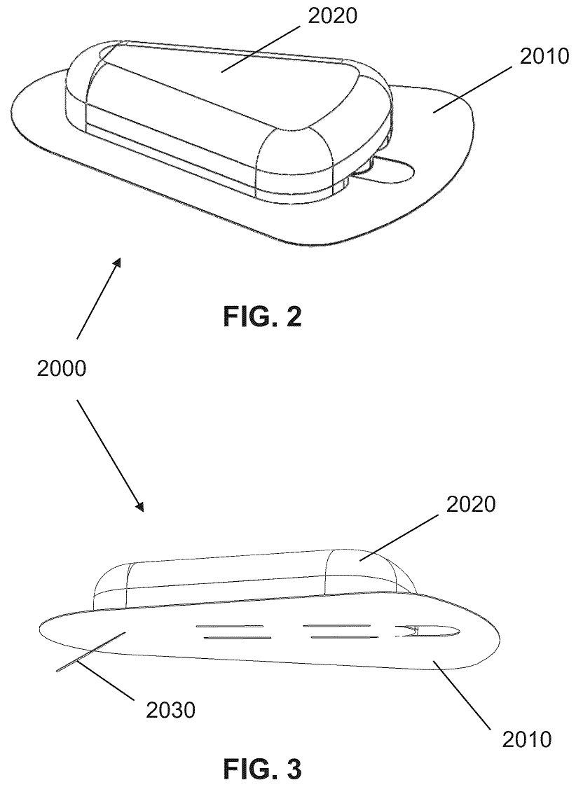

FIGS. 2 and 3 show different schematic views of an exemplary analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 2030 which may include an integrated analyte sensing region such as a glucose sensor.

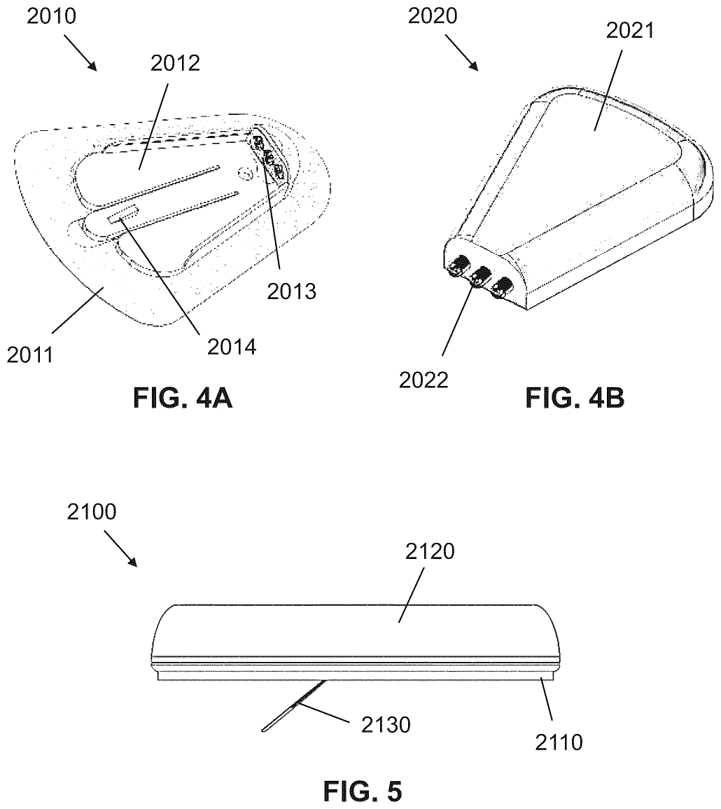

FIG. 4A shows the base or cradle 2010 of the monitor 2000. In the embodiment shown, the base or cradle 2010 is connectable and detachable from the transmitter 2020, but in other embodiments, the monitor may be a single integrated and connected unit. The cradle 2010 includes an adhesive or similar layer 2011 for connecting the cradle to the patient's skin. The adhesive layer may have a larger footprint or size compared to the rest of the cradle in order to facilitate a stronger connection to a patient or one that will last for a longer period of time, for example, multiple days or weeks. Other methods of attachment to a patient's skin may also be contemplated within the spirit and scope of the invention.

The cradle 2010 further includes a main body 2012 configured to facilitate attachment with the transmitter 2020. In the example shown, the body 2012 of the cradle is wedge-shaped to match a similar wedge shape of the transmitter 2020, but in other embodiments, the body and the transmitter may both be other shapes. In one region of the body, the cradle 2010 may further include contacts 2013 to facilitate an electrical connection with the transmitter 2020. In the embodiment shown, the contacts 2013 are located to one side of the cradle 2010, but in other embodiments, the contacts may be included in other regions of the cradle, based on the specific design and connection characteristics of the particular monitor. The cradle 2010 may also include a snap or other locking feature 2014 to facilitate a robust connection with the transmitter 2020 and reduce or eliminate occurrences of inadvertent detachment.

While not shown in FIG. 4A due to the angle of the perspective view, the cradle 2010 may further include the sensor member 2030 as an integrated component. For example, for at least some of the embodiments of applicator assemblies discussed below, the applicator assemblies are configured to attach cradles together with integrated sensors, so that actuation of the applicator assemblies will both deploy and properly position the sensor member under the patient's skin, as well as adhere the cradle to the outside of the patient's skin, in a single user step.

The base or cradle may further include other components, for example, at least some electronic circuitry to enable interconnectivity with the transmitter, and/or a battery. In particular, in some embodiments of the invention, the battery or other power source may be provided somewhere in the housing specifically to spatially separate the power source from the transmitter 2020 (discussed further below) and other electronics prior to assembly of the monitor, for example, during product manufacturing and shipping. In such embodiments, assembly of the transmitter with the cradle may close or otherwise complete an electrical circuit between the battery and the transmitter, as a step in electrically activating the monitor during assembly, discussed in greater detail below.

The transmitter 2020 may include a main transmitter body 2021. The transmitter body 2021 includes at least a transmitter, and may further include other components such as a circuit board, memory, or other components for the transmitter to function properly. The transmitter 2020 further includes contacts 2022 configured to engage with the contacts 2013 on the cradle 2010, in order for example, to create a closed circuit for the monitor 2000 to activate and function properly.

Like the cradle, the transmitter may also include other components, for example, a battery. It will be recognized that the specific design of the monitor will dictate arrangement of different components and whether they are found on the cradle or the transmitter. For example, if a battery is housed in the cradle, a second battery may not be needed to be housed in the transmitter. As further examples, in embodiments where the battery is housed together with the transmitter, activation of the battery in some embodiments may not involve closing or completing a circuit between the battery and the transmitter or other electronic components of the monitor, but may instead involve software processes that automatically initiate, for example, when a sensor is detected upon assembly between the transmitter and the cradle. In yet other embodiments, the battery, transmitter, and sensor may be arranged serially, for example, such that even though the battery and the transmitter are housed together, routing to the sensor may still be necessary to close or complete the circuitry for supplying power. Various other arrangements may also be contemplated without departing from the spirit or scope of the invention.

As mentioned above, other embodiments of analyte monitors may be integrated single-piece monitors. By way of example, FIG. 5 shows a side view of another exemplary monitor 2100 including an analyte sensor according to embodiments of the invention. Monitor 2100 is a one-piece monitor which includes a base 2110 that may have an integrated adhesive layer, a body 2120 which may include some or all of the components discussed above with respect to monitor 2000, and a sensor member 2030. More or less components may be needed for proper functionality of such a one-piece monitor. For example, separate contacts may not be needed in one-piece monitor designs. In such designs, where the components may generally be all housed together at the outset, automatic activation of the device may be achieved absent any physical assembly steps, for example, using solely software based on some triggering initiation step during application, such as detachment of an applicator if an applicator is used for applying the monitor to the patient. Various other activation steps may also be contemplated without departing from the spirit or scope of the invention.

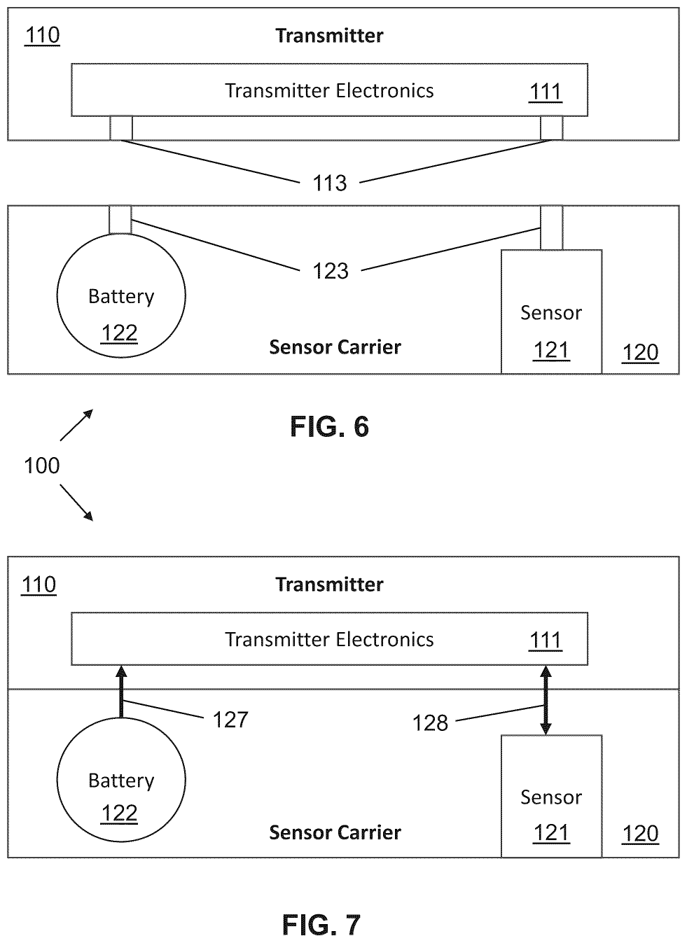

FIGS. 6 and 7 will now be discussed with respect to one example arrangement of an analyte monitor capable of being automatically activated, for example, upon physical assembly of separate components. As previously discussed, automatic activation of analyte monitors according to embodiments of the invention may be effected via physical assembly or hardware connectivity, software-based activation, or a combination of the two approaches, among other methods and combination of methods. Particularly, an objective of embodiments of the invention is for analyte monitors to be automatically activated, without requiring any specific actions by the end user to begin monitoring by the monitor. Specifically, for continuous glucose monitors, according to embodiments of the invention, a monitoring session automatically begins without any user interaction required. In this manner, the fastest possible activation and start-up time can be achieved, and readings and general monitoring can begin as soon as possible, without any setup by the user.

First, as seen in FIG. 6, a monitor 100 may include separate transmitter 110 and sensor carrier or other cradle or housing 120. The transmitter 110 may include general transmitter electronics 111, various other related or unrelated electronics such as a general controller, a PCB, or other circuitry. The transmitter 110 may further include electrical contacts 113 that are configured to engage corresponding electrical contacts on the sensor carrier 120. While only two contacts 113 are schematically illustrated, it should be understood that more or less contacts may be employed in other embodiments. For example, there are typically two, three, or more sensor contacts dedicated to electrically connecting the sensor 121 in the sensor carrier 120.

The sensor carrier 120 may include or have a separately attachable sensor 121, and may further include an integrated battery 122 and electrical contacts 123. Other components may also be held on the sensor carrier 120 in other embodiments.

The monitor 100 can be assembled, for example, by an end user, with a schematic representation of an assembled monitor 100 shown in FIG. 7. For example, an end user may first apply a sensor carrier 120 or other cradle or housing onto their skin. Here, the sensor 121 may be inserted under the user's skin using a variety of different methods, for example, via a separate applicator, while the rest of the sensor carrier 120 remains on top of the patient's skin, where for example an adhesive holds the sensor carrier 120 in place at a desired position.

The transmitter 110 may thereafter be attached to the sensor carrier 110, whereby the electrical contacts 113 of the transmitter 110 electrically connect with the corresponding electrical contacts 123 of the sensor carrier 120. This may cause, for example, the transmitter electronics 111 to receive power from the battery 122 via the connection 127. Furthermore, the transmitter electronics 111 may additionally be electrically connected to the sensor 121 via the connection 128 through initial assembly step. In some other embodiments, this assembly step may not directly be effected by the end user, but rather, for example, via an applicator (not shown) that may provide a one-step application approach, e.g., insertion of the sensor 121 under the skin, adhering of the sensor carrier 120 on the skin, and assembly of the transmitter 110 to the sensor carrier 120 all via a single actuation on the applicator. Here, assembly of the transmitter 110 to the sensor carrier 120 may happen during or after the insertion of the sensor 121, based on the mechanics of the particular applicator being used. Other embodiments may effect assembly in other ways.

In this embodiment, upon physical assembly of the respective parts, the transmitter 110, now powered, is configured to immediately apply a bias to the sensor 121 and to begin sampling and processing signals from the sensor 121. The device may further include storage means or other mechanism to retain sensing information from the sensor 121 upon activation, and prior to processing of the collected sensing information.

Thereafter, when convenient for the end user, the transmitter can then be paired with a user device, for example, a mobile device, where the sensing information can be sent and processed. Furthermore, any other required calibration inputs or initiation information can be applied at this later time. In this manner, the end user is not rushed to connect the monitor with an external device right after application/assembly of the monitor, and is not at risk of losing potentially important data between application and activation that may otherwise be lost in prior devices which required a manual activation.

Various different mechanical arrangements can be provided in different embodiments to achieve a similar automatic activation of the monitor. In the above example shown in FIGS. 6 and 7, the transmitter may be configured to activate immediately upon receiving power, when the transmitter is attached to the sensor carrier after the sensor has already been inserted, since the transmitter physically receives power from the battery after attachment to the sensor carrier. In another embodiment, the transmitter may be activated during insertion of the sensor, for example, if the insertion operation itself mechanically connects the transmitter to the sensor carrier, e.g., via a separate applicator. Under yet another different embodiment involving, for example, single disposable units similar to the one shown in FIG. 5, the transmitter may be activated automatically at insertion, if the insertion step connects the battery power to the electronics during insertion, for example, via a switch or if the sensor insertion otherwise effects electrical connectivity between different internal components.

In addition, similarly as previously discussed, automatic activation may not involve any physical electrical connections, but may instead be software based, for example, where activation is automatically triggered when the sensor is inserted under the patient's skin, or after a separate transmitter is attached to a base or other sensor carrier. Such arrangements may be advantageous, for example, in embodiments where the battery is housed together with the transmitter electronics, but where power-up of the transmitter is not desired until after the monitor has been fully assembled. In yet other embodiments, a combination of physical/mechanical and software steps may be arranged in combination to effect automatic activation of the monitor. Generally, embodiments of the invention are intended to cover devices where a further explicit activation step is not required by the end user after the sensor has been physically applied and fully assembled on the end user, either for example via a direct manual activation step or via connecting the assembled device to another device, e.g., remote pairing or wireless connection with a mobile device, to effect faster startup of the monitor and to reduce user error.

Embodiments of the invention improve on the prior art in many aspects. For example, monitors according to embodiments of the invention do not require detecting the presence of a sensor to start up or activate. An external device would no longer be required to activate and start a sensing/monitoring session. No battery power is consumed prior to the start of a session, so that shelf life of the device prior to application by an end user is maximized. And further, when used in a two-part continuous glucose monitor system similar to that shown in FIGS. 6 and 7, a durably manufactured transmitter, including many of the most expensive components of the monitor, can be reused multiple times and has no limit on operating life due to battery power limitations, so long as the battery is partitioned separately in disposable or otherwise replaceable sensor carriers, thereby potentially reducing the cost of using continuous glucose monitors significantly.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. An analyte monitor comprising:
a sensor configured to be inserted under a patient's skin;
a housing configured to be adhered to a surface of the patient's skin;
a transmitter for transmitting information associated with the sensor; and
a battery for providing power to the analyte monitor;
wherein the analyte monitor is not electrically activated prior to the sensor being inserted under the patient's skin and is automatically electrically activated after the sensor is inserted under the patient's skin.

2. The analyte monitor of claim 1, wherein the transmitter is separable from the housing.

3. The analyte monitor of claim 2, wherein the battery is housed in the housing.

4. The analyte monitor of claim 2, wherein the patient is configured to assemble the transmitter to the housing, and wherein the analyte monitor is automatically electrically activated upon assembly of the transmitter to the housing.

5. The analyte monitor of claim 4, wherein the battery is housed in the housing, and wherein the automatic electrical activation comprises electrically connecting the battery to the transmitter to provide power to the transmitter.

6. The analyte monitor of claim 1, wherein the housing holds the battery and at least part of the sensor, while the transmitter is configured to be spaced apart from the housing.

7. The analyte monitor of claim 1, wherein the automatic electrical activation is configured to be effected absent any further actions by the patient.

8. A method of operating an analyte monitor comprising a sensor, a transmitter, and a battery, the method comprising:
inserting the sensor under a patient's skin; and
connecting the analyte monitor with an external device;
wherein operation of the analyte monitor is automatically activated after the sensor is inserted under the patient's skin and prior to the analyte monitor being connected with the external device.

9. The method of claim 8, wherein the operation of the analyte monitor is automatically activated absent any explicit activation step by the patient.

10. The method of claim 8, wherein the connection is a wireless connection.

11. The method of claim 8, wherein the analyte monitor is separated into at least two parts, and wherein the method further comprises assembly of the at least two parts together.

12. The method of claim 11, wherein a first part of the at least two parts comprises a housing that holds the battery and at least part of the sensor, and wherein a second part of the at least two parts comprises the transmitter.

13. The method of claim 12, wherein the assembly of the at least two parts closes an electrical connection between at least the battery and the transmitter.

14. The method of claim 13, wherein the assembly of the at least two parts further electrically connects the transmitter with the sensor.

15. The method of claim 12, wherein the second part comprising the transmitter is configured to be detached from the first part and to be reused with a first part of a different analyte monitor.

16. The method of claim 8, wherein the operation of the analyte monitor comprises collecting data from the sensor prior to the analyte monitor being connected with the external device.

17. The method of claim 16, wherein the analyte monitor further comprises storage for storing the collected data from the sensor prior to the analyte monitor being connected with the external device.

18. The method of claim 16, wherein the collected data is delivered to the external device after the analyte monitor is connected with the external device.
